(19) United States
(12) Patent Application Publication
(10) Pub. No.: US 2021/0001016 A1
(43) Pub. Date: Jan. 7, 2021

(54) RADIOPAQUE AND ECHOGENIC COATINGS FOR MEDICAL DEVICES

(71) Applicant: ADVANSOURCE BIOMATERIALS, Wilmington, MA (US)

(72) Inventors: Khristine CARROLL, North Andover, MA (US); Xiaoxi Kevin CHEN, Natick, MA (US)

(73) Assignee: MITSUBISHI CHEMICAL PERFORMANCE POLYMERS, INC., Warren, MI (US)

(21) Appl. No.: 16/969,462

(22) PCT Filed: Feb. 14, 2019
(86) PCT No.: PCT/US2019/017924

§ 371 Date: Aug. 12, 2020

Related U.S. Application Data
(60) Provisional application No. 62/630,334, filed on Feb. 14, 2018.

(51) Int. Cl. A61L 31/12; A61L 31/10; A61L 31/02; A61L 31/18

(52) U.S. Cl. CPC A61L 31/128; A61L 31/10; A61L 2420/04; A61L 31/18; A61L 2420/02; A61L 31/022

(57) ABSTRACT

The present invention discloses methods for producing coatings for medical devices that are both echogenic and radiopaque.

### CROSS-REFERENCE TO RELATED APPLICATION

This application claims priority of U.S. Provisional Patent Application No. 62/630,334, filed Feb. 14, 2018, the entire contents of which are incorporated by reference herein.

### FIELD OF THE INVENTION

The present invention discloses methods for producing coatings for medical devices that are both echogenic and radiopaque.

### BACKGROUND OF THE INVENTION

Ultrasound has been widely used to guide needle, catheter and guidewire placement and for vascular access, nerve blockade, drainage of pleural or ascitic fluid collections and percutaneous tracheostomy. Ultrasound allows identification of the target and collateral structures and real-time guidance to precisely place needles and other inserted devices.

The visibility of a needle or other inserted devices in ultrasound guided procedures is extremely important. Without accurate identification of the position of the needle it is possible that damage to collateral structures may occur. However, most medical devices have an acoustic impedance similar to that of the tissue into which the device is inserted. Consequently, visibility of the device can be poor and accurate placement can become extremely difficult.

Radiopaque materials, such as bismuth subcarbonate and barium sulfate, have been widely used in the design of various devices such as guidewires or stents that are used during radiological intervention. The radiopacity of a given endovascular device is important since it allows the device to be tracked during the interventional procedure.

Due to the high density of radiopaque materials, it is possible to use them to provide a contrast for ultrasound visibility. Furthermore, it is advantageous to provide coatings for medical devices that are both echogenic and radiopaque.

### SUMMARY OF THE INVENTION

In light of the foregoing, the present invention provides medical device coatings that are both echogenic and radiopaque. More specifically, the coating composition of the present invention comprises a polymer matrix dispersed with radiopaque materials. The polymers used in the coating composition preferably adhere strongly to the substrate surfaces and allow a homogeneous dispersion of the radiopaque materials.

### BRIEF DESCRIPTION OF THE FIGURES

FIG. 1 is a drawing representing a substrate coated using subject invention echogenic and radiopaque coating comprising a polymer matrix and a dispersion of radiopaque materials.

FIG. 2 shows the comparison of 2 ultrasonograms. The one on the left corresponds to the ultrasonogram of an uncoated stainless-steel needle immersed in water. The one on the right corresponds to the ultrasonogram of a coated stainless-steel needle immersed in water. The coated stainless-steel needle was prepared using the echogenic and radiopaque coating of the subject invention, as described in Example A.

### DETAILED DESCRIPTION OF THE INVENTION

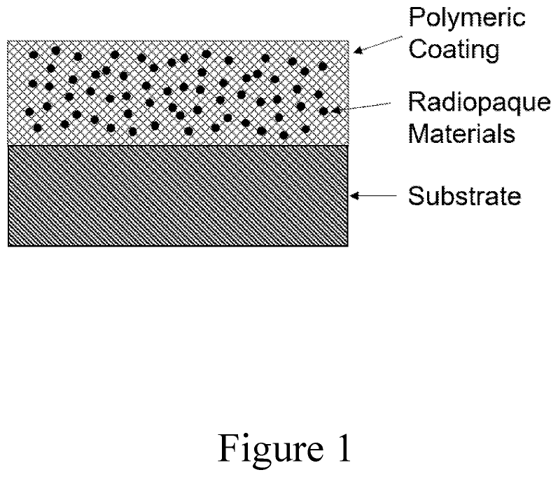

With reference to FIG. 1, the substrate, which is the outer surface of a needle or other medical devices, is coated with a matrix formed by polymer and/or other materials dispersed with radiopaque materials.

The polymers used to form the polymer matrix preferably are biocompatible and have good tensile strength and adhesion to a wide array of metallic and polymeric substrates. Suitable polymers include those that have been used as polymeric coatings for medical devices such as polyurethane (PU), polymethylmethacrylate (PMMA), poly vinylalcohol (PVA), poly-N-vinylpyrrolidone (PVP), polyethylene oxide (PEO), and copolymers thereof. Mixtures and blends of these polymers also can be used. Other matrix based coatings or jackets can also be used.

Radiopaque materials used to be dispersed in the polymer matrix preferably are biocompatible. Suitable radiopaque materials include, but not limited to, bismuth subcarbonate, bismuth oxide, bismuth oxychloride, and barium sulfate.

The radiopaque materials and the polymers can be mixed together in one or more organic solvents to provide a coating composition. Suitable solvents that can be used include, but not limited to, tetrahydrofuran, acetone, methylethylketone, dimethylformamide, dimethyacetamide, ethylene carbonate, propylene carbonate, diglyme, N-methylpyrrolidone, ethyl acetate, ethylene and propylene glycol diacetates, alkyl ethers of ethylene and propylene glycol monoacetates, toluene, xylene and sterically hindered alcohols such as t-butanol and diacetone alcohol. In preferred embodiments, the organic solvent or solvent mixture is evaporative. For example, tetrahydrofuran can be used. The total solid loading can be between about 5 wt. % and about 30 wt. %, where the loading of the radiopaque materials is between about 10 wt. % and about 200 wt. % of that of the polymer.

To improve the echogenicity of a medical device, at least a portion of the surface of the medical device can be coated with the present coating composition. Various coating techniques such as spin coating, drop-casting, zone casting, dip coating, blade coating, and spraying can be used, depending on the shape of the medical device. For example, the medical device can be an elongated member such as a catheter, a guidewire, or a needle, or a planar or spherical member such as an implant or a balloon. Typical thickness of the coating can range from about 0.01 mm to about 0.2 mm. The thickness achieved by one application of the coating composition will depend on the viscosity of the coating composition, the coating method, as well as the speed and the temperature at which the coating is applied. In some embodiments, multiple applications of the coating may be needed to build up the required thickness. The coating is then allowed to dry.

### EXAMPLES

### Example A

Stainless-steel needles were coated with the subject invention method. A coating solution was prepared by first dissolving 5% (w/v) ChronoFlex AL in tetrahydrofuron, followed by mixing 10% (w/v) bismuth subcarbonate in the ChronoFlex solution until a homogeneous solution is obtained. Stainless-steel needles were then dipped into the coating solution and lifted up slowly. The stainless-steel needles were then dried at room temperature for 30 minutes.

### Example B

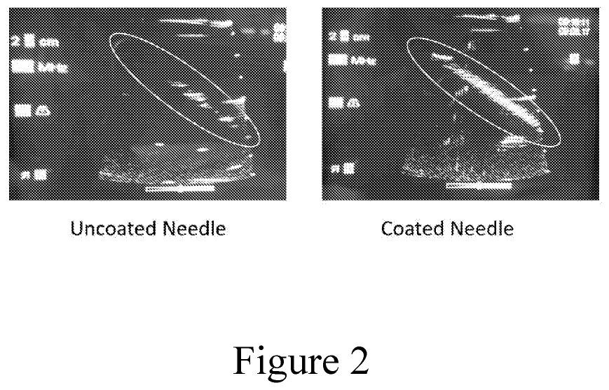

Stainless-steel needles prepared using the durable echogenic coating of the subject invention as described in Example A were compared with uncoated stainless-steel needles for ultrasound visibility in water. FIG. 2 shows the comparison of 2 ultrasonograms. The one on the left corresponds to the ultrasonogram of an uncoated stainless-steel needle immersed in water. The one on the right corresponds to the ultrasonogram of a coated stainless-steel needle immersed in water. The coated needle has significantly improved ultrasound visibility compared to the uncoated needle.

The present teachings can be embodied in other specific forms without departing from the spirit or essential characteristics thereof. The foregoing embodiments are therefore to be considered in all respects illustrative rather than limiting on the present teachings described herein. The scope of the present teachings is thus indicated by the appended claims rather than by the foregoing description, and all changes that come within the meaning and range of equivalency of the claims are intended to be embraced therein.

What is claimed is:

1. A medical device comprising a coating for improving ultrasound visibility, wherein the coating comprises radiopaque materials dispersed within a polymer matrix.

2. A medical device of claim 1, wherein the polymer matrix includes polyurethanes.

3. A medical device of claim 1, wherein the polymer matrix includes polycarbonate-based urethanes.

4. A medical device of claim 1, wherein the polymer includes silicones.

5. A medical device of claim 1, wherein the radiopaque materials include bismuth subcarbonate.

6. A medical device of claim 1, wherein the radiopaque materials include bismuth oxide.

7. A medical device of claim 1, wherein the radiopaque materials include bismuth oxychloride.

8. A medical device of claim 1, wherein the radiopaque materials include barium sulfate.

9. A medical device of claim 1, wherein the coating is prepared by dipping the substrate in the coating solution containing the matrix and the radiopaque materials.

10. A medical device of claim 9, wherein the coating solution contains 0.1-20% (weight to volume) of the combined matrix and radiopaque materials.

11. A medical device of claim 9, wherein the solvent is either tetrahydrofuran, dimethylacetamide, or a mixture of both.

12. A medical device of claim 9, wherein a multiple dipping process is used to obtain the coating.
